(19) United States
(12) Patent Application Publication
(10) Pub. No.: US 2020/0140957 A1
(43) Pub. Date: May 7, 2020

(54) METHOD FOR DETERMINING THE SUSCEPTIBILITY OF A PATIENT SUFFERING FROM PROLIFERATIVE DISEASE TO TREATMENT USING AN AGENT WHICH TARGETS A COMPONENT OF THE PD I/PD-LI PATHWAY

(71) Applicant: Oncologica UK Ltd., Cambridge Cambridgeshire (GB)

(72) Inventors: Marco Loddo, Cambridge Cambridgeshire (GB); Gareth Williams, Cambridge Cambridgeshire (GB); Keeda Snelson, Cambridge Cambridgeshire (GB)

(21) Appl. No.: 16/622,419

(22) PCT Filed: Jun. 13, 2018
(86) PCT No.: PCT/GB2018/051624

§ 371 Date: Dec. 13, 2019

(30) Foreign Application Priority Data
Jun. 13, 2017 (GB) 1709360.0
Oct. 25, 2017 (GB) 1717512.6

(51) Int. Cl. C12Q 1/6886; G16H 50/20; G16H 20/10

(52) U.S. Cl. CPC C12Q 1/6886; G16H 50/20; G16H 20/10; G01N 2800/52; C12Q 2600/156; C12Q 2600/158; C12Q 2600/106

(57) ABSTRACT

A method for determining the susceptibility of a patient suffering from proliferative disease to treatment using an agent which targets a component of the PD1/PD-L1 pathway, said method comprising determining in a tumour sample from said patient at least three biomarkers selected from list including those directly related to the PD-1/PD-L1 pathway as well as other oncogenic biomarkers and the tumour mutational burden, and relating the presence of more than one of said biomarkers as an indicator susceptibility to said agent. Methods for making the determinations and algorithms for dealing with the results are also described and claimed.

The present invention relates to a method for determining the susceptibility of a patient suffering from proliferative disease, such as cancer, to treatment using particular types of agent. It further comprises the development of treatment regimens for selected patients, based upon the determination, kits for carrying out the determination and computers programmed to carry out the determination.

### BACKGROUND TO THE INVENTION

Anti-PD-1/PD-L1 directed immunotherapies have become one of the most important group of agents used in immunotherapy. The PD-1/PD-L1 pathway is normally involved in promoting tolerance and preventing tissue damage in the setting of chronic inflammation. Programmed death 1 (PD-1) and its ligands, PD-L1 and PD-L2, deliver inhibitory signals that regulate the balance between T cell activation, tolerance, and immunopathology. The PD-1 programmed death-ligand 1 (PD-L1) is a transmembrane protein that binds to the programmed death-1 receptor (PD-1) during immune system modulation. This PD-1/PD-L1 interaction protects normal cells from immune recognition by inhibiting the action of T-cells thereby preventing immune-mediated tissue damage.

Harnessing the immune system in the fight against cancer has become a major topic of interest. Immunotherapy for the treatment of cancer is a rapidly evolving field from therapies that globally and non-specifically stimulate the immune system to more targeted approaches. The PD-1/PD-L1 pathway has emerged as a powerful target for immunotherapy. A range of cancer types have been shown to express PD-L1 which binds to PD-1 expressed by immune cells resulting in immunosupressive effects that allows these cancers to evade tumour destruction. The PD-1/PD-L1 interaction inhibits T-cell activation and augments the proliferation of T-regulatory cells (T-regs) which further suppresses the effector immune response against the tumour. This mimics the approach used by normal cells to avoid immune recognition. Targeting PD-1/PD-L1 has therefore emerged as a new and powerful approach for immunotherapy directed therapies.

Targeting the PD-1/PD-L1 pathway with therapeutic antibodies directed at PD-1 and PD-L1 has emerged as a powerful therapy in those cancer types displaying features of immune evasion. Disrupting the PD-1/PD-L1 pathway with therapeutic antibodies directed against either PD-1 or PD-L1 (anti-PD-L1 or anti-PD-1 agents) results in restoration of effector immune responses with preferential activation of T-cells directed against the tumour.

A range of cancer types including, melanoma, renal cell carcinoma, lung cancers of the head and neck, gastrointestinal tract malignancies, ovarian cancer, haematological malignancies are known to express PD-L1 resulting in immune evasion. Anti-PD-L1 and anti-PD-1 therapy has been shown to induce a strong clinical response in many of these tumour types, for example 20-40% in melanoma and 33-50% in advanced non-small cell lung cancer (NSCLC). A number of these antibodies, for example anti-PD-1 directed agents Nivolumab and Pembrolizumab, have now received FDA-approval for the treatment of metastatic NSCLC and advanced melanoma.

There are nine drugs in development targeting the PD-1/PD-L1 pathway, and the current practice of pharmaceutical companies is to independently develop an anti-PD-L1 immunohistochemical (IHC) diagnostic assays as a predictor of response to anti PD-1/anti PD-L1 directed therapies. These PD-1/PD-L1 directed therapies include Pembrolizumab, atezolizumab, avelumab, nivolumab, durvalumab, PDR-001, BGB-A317, REG W2810, SHR-1210 (Table 1 hereinafter).

The leading Biopharma companies have all chosen an immunohistochemical approach on paraffin wax embedded formalin fixed diagnostic biopsies and resection tissues/samples (PWET) for the development of companion diagnostics for anti-PD-1/PD-L1 directed therapies. All these tests involve the application of a monoclonal antibody raised against PD-L1 applied to the tissue section using a standard immunohistochemical assay approach with enzyme linked chromogen detection systems. The immunohistochemical staining of cells, either partial of complete surface membrane staining for PD-L, is then assessed manually by microscopic examination by a pathologist to determine the proportion of cells which express PD-L1. This is then reported a tumour proportion score. Some assays assess only the tumour cell expression of PD-L1, others assess both tumour cells and the expression of PD-L1 in the associated intratumoural and peritumoural immune cell infiltrates (ICs). The tumour proportion score is defined as the percentage of viable tumour cells showing partial or complete membrane staining (≥1+) relative to all viable tumour cells present in the sample (positive and negative).

Representative examples of companion diagnostic assays assessing either the tumour proportion score or the tumour proportion score and immune cell infiltrate score for PD-L1 are shown below (Table 2 hereinafter). These tests have been developed by DAKO and Ventana highlighting the two types of approaches.

There are major problems associated with the current IHC PD-L1 based companion diagnostics for anti-PD-1/PD-L1 directed anti-cancer immunotherapies.

For instance, to date each therapeutic drug has been approved in conjunction with separate companion diagnostic tests using different PD-L1 or PD-1 antibodies. Antibodies can vary vastly in their sensitivity and specificity. The matrix of therapeutics and diagnostics therefore presents a complex challenge for testing and decision-making in the clinic. In addition, the purpose of each assay has been shaped by clinical experience. As a result, the PD-L1 IHC 22C3 pharmDx, which was used as an inclusion criteria for patient enrichment in advance NSCLC trials with pembrolizumab, is required for clinical use of the drug in this indication, whereas the PD-L1 IHC 28-8 pharmDx, retrospectively evaluated in the same patient population, is used to inform on the risk-benefit assessment for different patient subgroups as defined by the biomarker positively.

The present assays do not include analysis of the PD-L2 ligand, which is also relevant in the PD-1 pathway.

Compounding the issues with the current PD-L1 IHC assays, evaluating PD-L1 in the absence of PD-1 and PD-L2 analysis compromises integrated assessment of the PD1/PD-L1/PD-L2 signalling network which reduces the accuracy of precisely identifying the patients likely to respond to anti PD-1/PD-L1 directed therapy. Similarly, PD-1 testing on its own lacks the predictive information provided by PD-L1 and PD-L2 analysis.

The PD-L1 signalling axis involves other major components in addition to PD-1 and PD-L1 which have been shown to be predictors of response to anti-PD-1/PD-L1/PD-L2 directed immunotherapy agents including increased expression levels of NFATC1, PIK3CA, PIK3CD, PRDM1, PTEN, PTPN11, MTOR, HIF1A, FOXO1. It is not possible to analyse such a broad spectrum of aberrant gene expression using conventional IHC.

Notably a wide range of oncogenic mutations have also been shown to be major predictors of response to anti-PD-1/PD-L1/PD-L2 directed immunotherapies. These include hot spot mutations in oncogenes and oncogenic chromosomal rearrangement events leading to fusion genes. Again IHC approaches cannot be used to assess such a broad range of oncogenic genetic events as predictive biomarkers of response for anti-PD-1/PD-L1/PD-L2 directed immunotherapies

Aberrant overexpression of PD-L1 and PD-L2 can also occur as a result of gene amplification. The IHC based approach is unable to detect gene amplification of the PD-L1 and PD-L2 ligands. Since a number of oncogenes can undergo amplification which is predictive of response to anti-PD-1/PD-L1/PD-L2 directed immunotherapies, these are not detectable using an IHC approach.

Furthermore, mutations in the coding sequence of PD-L1 is a predictor of response to anti-PD-1/PD-L1/PD-L2 directed immunotherapies but cannot be detected by IHC.

The cellular, spatial, and temporal heterogeneity of PD-L1, PD-L2 and PD-1 expression combined with the problems discussed above all contribute to the poor prediction accuracy of these biomarkers in the clinic (i.e. lack of both positive and negative predictive values). Moreover, it has been shown that a proportion of tumours scored positive by IHC for PD-L1, PD-L2 and PD-1 fail to respond to anti-PD-1/ PD-L1/PD-L2 directed immunotherapies and similarly a subset of patients whose tumour biopsies are PD-L1 positive with respect to PD-L1 protein expression actually achieve little clinical benefit. This again suggests the IHC based assays are inadequate resulting in the generation of false positive and negative results.

A comprehensive analysis of the PD-1/PD-L1/PD-L2 immunoregulatory pathway requires analysis of all cellular components in the tumour involved in the anti-tumour immune response. This includes tumour cells, T immune cells and antigen presenting cells (APSs) the latter alternatively termed macrophages or dendritic cells. However most of the available assays assess tumour cells only.

The microscopic examination by individual pathologists of PD-L1 immunostaining varies according to their training and experience. There is therefore major inter-observer variability in the generation of a tumour proportion score for PD-L1. Moreover, the assessment of a labelling index is extremely difficult as highlighted in high levels of intra-variability by a single pathologist.

Current IHC based assays use different antibody clones which vary in sensitivity and specificity and interpretation is carried out by different pathologists who vary in their interpretation of tumour proportion score and staining intensity. This also applies to assessment of immune cells for PD-L1 expression. This further confounded by different tissue architecture associated with different tumour types. Taken together, these problems make standardisation across laboratories very difficult.

The problems for pathologists generating accurate labelling indices for IHC biomarkers is highlighted in earlier studies in trying to develop labelling index protocols for Ki67. Notably automated image analysis has also failed to address this problem.

In addition, selection of antibody clones, optimal cut-off points standardisation of PD-L1 staining is problematic and has not yet been established. Finally, the IHC PD-L1 based tests are fundamentally semi-quantitative in nature and therefore lack the precision provided by quantitative assays.

Genome-wide mutation analysis (mutanome) using next generation sequencing technology and diversity analysis of the T-cell or B-cell repertoire (Immunome) has been reported as generating attention as a potential strategy for identifying predictive biomarkers (Y. Iwai et al. Journal of Biomedical Science (2017) 24:26 DOI 10.1186/s12929-017-0329-9). However, the it has been recognised that the choice of biomarkers is important and that individual biomarkers do not always correspond to a high response according to cancer type.

### SUMMARY OF THE INVENTION

According to the present invention there is provided a method for determining the susceptibility of a patient suffering from proliferative disease to treatment using an agent which targets a component of the PD1/PD-L1 pathway, said method comprising determining in a tumour sample from said patient at least three biomarkers selected from the group set out in Tables 3A-3D hereinafter, and relating the presence of more than one of said biomarkers as an indicator susceptibility to said agent.

As used herein, the term “biomarker” refers to any molecule, gene, sequence mutation or characteristic such as increased or decreased gene expression, which is indicative of an aberration in the PD1/PDL1 pathway. These may include mutations in the gene sequence, in particular ‘hotspot’ mutations which are known to give rise to oncological outcomes, copy number variations of genes, aberrant gene fusions or increased or decreased RNA expression.

An agent which targets a component of the PD1/PD-L1 pathway will suitably be an agent which targets or binds to PD1, PD-L1 or PD-L2 proteins, although agents which target the expression of such proteins, for example, DNA or RNA encoding those proteins may also be envisaged.

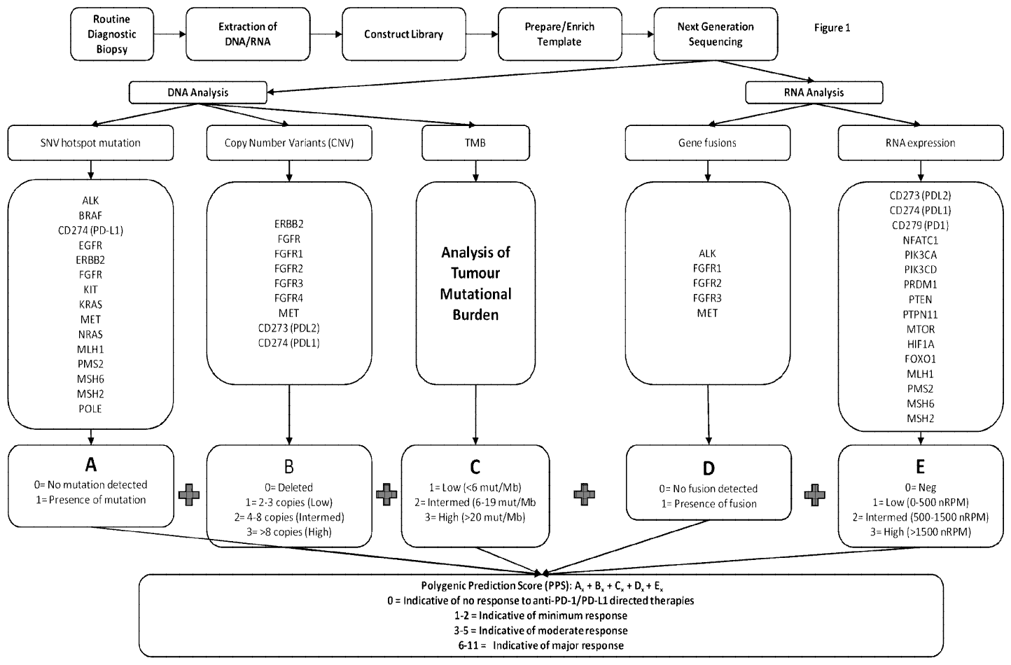

In particular genes which may be used as biomarker for use in the method of the invention, and the changes associated with a cancer risk are set out in Tables 3A-3D and FIG. 1 hereinafter.

In a particular embodiment, the method of the invention will involve the determination of at least 5, for instance at least 8 such as at least 10 of the biomarkers in Table 1, and preferably all of the biomarkers in Table 1. In particular, the larger the number of biomarkers used, the greater the probability that dysregulated genes will be identified, so that false negatives (i.e. where susceptible patients are missed), are avoided.

In a particular embodiment, at least one of the biomarkers detected is directly associated with the PD-1/PD-L1 pathway, and so is a biomarker of a gene selected from CD279(PD1), CD274(PD1) or CD273(PD2). In a particular embodiment, both PD-L1 and PD-1 are assessed together providing a much more powerful assessment of the PD-1/PD-L1 signalling axis.

In another particular embodiment, the method measures both PD-L1 and PD-L2 gene amplification (copy number variant; CNV) which has been linked to mRNA overexpression and may represent a much more reliable parameter to predict response to PD-1/PD-L1 inhibitors.

In a particular embodiment, the method of the invention analyses and integrates PD-1 and PD-L1 expression in all cell populations involved in the tumour-Immune cell interaction including tumour cells, immune cells and antigen presenting cells (APCs).

In a particular embodiment, at least one of the genes detected is not directly associated with the PD1/PDL-1 pathway but is a biomarker listed in Tables 3A-3D, which is other than CD279 (PD1), CD274 (PD1) or CD273 (PD2). By selecting a range of biomarkers associated with different functions, the applicants have appreciated that a better indication of susceptibility to treatment which targets an immune pathway and in particular, the PD-1/PD-L1. Mutations in other genes, in particular oncogenic mutations, are likely to give rise to so-called ‘neo-antigens’. Neo-antigens are mutated forms and in particular cancer-specific antigens, which can result in T-cell activation against cancer cells if the immune system is effective and not subject to suppression. Therefore, where neo-antigens are present, patients may show a more efficient and durable response to agents which act on immune pathways such as the PD-1/PD-L1 pathway.

In another embodiment, a further biomarker measured in accordance with the method of the invention is the tumour mutational burden (TMB). Unlike protein-based biomarkers, TMB is a quantitative measure of the total number of mutations per coding area of a tumour genome.

Since only a fraction of somatic mutations gives rise to neo-antigens, measuring the total number of somatic mutations (TMB) within a particular coding area acts as a proxy for neo-antigen burden. The TMB may be measured using exome sequencing, in particular using Next Generation Sequencing in particular of 411 genes covering a 1.7 Mb coding region. From all variants detected including indels, substitutions, etc., all likely germline polymorphisms and predicted oncogenic drivers are removed from the analysis. The latter is performed to prevent ascertainment bias of sequencing known cancer genes. Tumour Mutational Burden is then calculated as mutations per Mb of DNA sequenced (mut/MB). Analysis of TMB is both quantitative and qualitative and is reported as a metric (mut/Mb) as well as status. Status of High is classified as >20 mut/Mb, Intermediate 6-19 mut/Mb and Low <6 mut/Mb.

It has been recognised that TMB is a predictor of response to anti-PD-1/PD-L1/PD-L2 checkpoint inhibitors, and therefore it will provide an additional biomarker useful in the method of the invention. Proliferative disease which may be treated by such agents include cancer, in particular solid cancers such as FAdrenal Cancer, Anal Cancer, Basal and Squamous Cell Skin Cancer, Bile Duct Cancer, Bladder Cancer, Bone Cancer, Brain and Spinal Cord Tumours, Breast Cancer, Cancer of Unknown Primary, Cervical Cancer, Colorectal Cancer, Endometrial Cancer, Esophagus Cancer, Ewing's sarcoma, Eye Cancer, Gallbladder Cancer, Gastrointestinal Carcinoid, Gastrointestinal Stromal Tumour (GIST), Kidney Cancer, Laryngeal and Hypopharyngeal Cancer, Liver Cancer, Lung Cancer, Lung, Carcinoid Tumour, Malignant Mesothelioma, Melanoma, Merkel Cell Skin Cancer, Nasal Cavity and Paranasal Sinuses Cancer, Nasopharyngeal Cancer, Neuroblastoma, Non-Small Cell Lung Cancer, Oral Cavity and Oropharyngeal Cancer, Osteosarcoma, Ovarian Cancer, Pancreatic Cancer, Penile Cancer, Pituitary Tumours, Prostate Cancer, Retinoblastoma, Rhabdomyosarcoma, Salivary Gland Cancer, Skin Cancer, Small Cell Lung Cancer, Small Intestine Cancer, Soft Tissue Sarcoma, Stomach Cancer, Testicular Cancer, Thymus Cancer, Thyroid Cancer, Uterine Sarcoma, Vaginal Cancer, Vulvar Cancer.

The methodology used to determine the particular biomarkers will vary depending upon the nature of the biomarker, and will be generally understood in the art. Where the biomarker gene expresses a protein or peptide, and in particular, a variant protein or peptide, these may be identifiable using immunoassay techniques such as ELISA.

However, in a particular embodiment, the biomarker is identified using DNA or RNA analysis or a combination thereof. In a particular embodiment, a tumour biopsy sample, such as a routine diagnostic PWET sample, from a patient suffering from cancer is obtained and nucleic acids (DNA and/or RNA) extracted from it. This is then used to construct a library, using conventional methods, for example as outlined below. The library is enriched as necessary and then used as a template for enrichment, again using conventional methods as outlined below. Analysis of this is then carried out using semiconductor Next Generation sequencing techniques, such as available from Oncologica UK Ltd.

In a particular embodiment the method of the invention uses targeted semiconductor sequencing to cover the entire coding regions of the biomarkers of interest. Amplicons have been designed to overlap for sequence coverage redundancy and to be able to amplify fragmented DNA templates obtained from routine diagnostic PWET samples. The method is therefore able to identify a wide variety of actionable genetic variants including point mutations, deletions, duplications, and insertions.

Thus, in a particular embodiment, the method of the invention utilises Next Generation Sequencing technology to quantitatively measure PD-1 and PD-L1 RNA expression levels from a sample, such as a single 10 μm section taken from routine formalin fixed paraffin embedded tumour samples. Thus the method of the invention can be carried out using only a small amount (<10 ng) of PWET material and it may be optimised for analysis of degraded DNA/RNA.

The method can be used to provide a quantitative test that gives a much more accurate method than immunohistochemistry (IHC) to determine those patients most likely to respond to anti-PD-1 and anti-PD-L1 directed therapies. By avoiding the use of antibodies, the problems associated with sensitivity and specificity of different antibody clones are avoided.

Some biomarkers may be most readily identified by an analysis of gene expression, for example using quantitative measurement of RNA transcripts. Thus in a particular embodiment, the method includes the step of analysing levels of RNA, wherein a change in the expression level as compared to wild type is indicative of the PD-1/PD-L1 pathway. Biomarkers which may be identified in this way are listed in Table 3A below and in FIG. 1. In particular, in tumours where such a pathway is implicated, elevated levels of RNA from CD273 (PD-L2), CD274 (PD-L1) and CD279 (PD-1) may be detected.

In a particular embodiment, gene expression at multiple exon-intron loci across PD-L1 and PD-1 mRNAs is carried out, which is then coupled to a bioinformatics programme that normalises gene expression across the whole gene allowing very accurate quantitative measurement of PD-1 and PD-L1 RNA expression levels.

In addition, elevated levels of RNA of NFATC1 (Nuclear Factor Of Activated T-Cells 1), PIK3CA (Phosphatidylinositol-4,5-Bisphosphate 3-Kinase Catalytic Subunit Alpha) PIK3CD (Phosphatidylinositol-4,5-Bisphosphate 3-Kinase Catalytic Subunit Delta), PRDM1 (PR domain zinc finger protein 1), PTEN, (Phosphatase and tensin homolog), PTPN11 (Tyrosine-protein phosphatase non-receptor type 11), MTOR (mechanistic target of rapamycin), HIF1a (Hypoxia-inducible factor 1-alpha) and FOXO1m (forkhead box class 01 mutant) may be quantified. In addition, the method of the invention may detect loss of gene expression of the mismatch repair genes MLH1, PMS2, MSH6 and MLH2. Loss of function of one of these genes results in genomic instability leading to increased expression of tumour surface neo-antigens and thereby increases response rates to anti-cancer directed immunotherapies.

Levels of any specific RNA which would be considered to be elevated as compared to normal and therefore indicative of a positive biomarker are shown in Table 3A.

In another embodiment, DNA from said sample is analysed and a mutation in a gene encoding a biomarker is detected that impacts on expression or function of the gene or gene product. Particular examples where mutation, for example via an SNV hotspot mutation occurs, are found biomarkers listed in Table 3B below. In particular, such biomarkers may be found in genes selected from the group consisting of ALK (anaplastic lymphoma kinase gene), BRAF (B-Raf gene), CD274 (PD-L1 gene), EGFR (epidermal growth factor receptor gene), ERBB2 (Receptor tyrosine-protein kinase erbB-2 or human epidermal growth factor receptor 2 gene), FGFR (fibroblast growth factor receptor gene), KIT (KIT proto-oncogene receptor tyrosine kinase gene), KRAS (K-ras gtpase gene), MET (MET proto-oncogene, receptor tyrosine kinase) or NRAS (N-ras protooncogene, GTPase gene). Specific mutations of these genes which are indicative of an oncogenic mutation are set out in Table 3B below. These mutations may be detected using conventional techniques, as illustrated hereinafter

In a particular embodiment, biomarkers resulting from gene rearrangement leading to aberrant gene fusions may be detected, and these are listed in Table 3C below. For example, it is recognised that the ALK gene may be fused with portions of the echinoderm microtubule-associated protein-like 4 (EML4) gene in some cancers, that FGFR genes may form fusions for instance with kinases in others, and that MET gene may become fused with other genes including for example TFG, CLIP2 or PTRZ1. Detection of any such fusion genes/proteins will provide a positive biomarker indication.

In another embodiment, the analysis identifies the presence of copy number variants which may lead to increased expression. In particular, DNA from said sample is analysed and the presence of a variation in copy number of a gene encoding a biomarker is detected. Suitable biomarkers in this case are selected from the group consisting of ERBB2, FGFR, FGFR1, FCFR2, FGFR3, FGFR4, CD273 (PD-L2 gene) or CD273 as listed in Table 3D. In such cases, an increase in copy number, which can result in amplification or increased expression is noted. In such cases, the greater the increase in copy number the higher the susceptibility indicated.

In these cases, normal copy numbers are summarised in Table 3D. Therefore, departures from these figures will be indicative of susceptibility to agents which target the PD-1/PD-L1 pathway.

The various analytical methods and techniques for the determination of the biomarkers are suitably carried out in a high-throughput assay platform as far as possible.

In a particular embodiment, an algorithm indicative of the level of susceptibility is applied to the results obtained as described above. In particular, a score of ‘0’ is applied to results which show no or minimal changes over wild type or normal expression profiles of the various biomarkers (e.g. 0-500 normalised Reads per Million Reads (nRPM) in the case of RNA expression), whereas a score of 1 is applied to any mutations or variations noted. In the case of multiple copies of a particular gene being detected, a higher score may be allocated depending upon the number of copies detected and a higher score may also be applied in the case of very high RNA expression level changes (e.g. >1500 nRPM). The TMB score discussed above may be included in such an algorithm. In this case, a score of 1 may be allocated for a ‘low’ TMB of <6 mut/Mb, a score of 2 may be allocated for an intermediate TMB of from 6-19 mut/Mb and a score of 3 may be allocated for a high TMB of >20 mut/Mb.

A particular example of such an algorithm is shown in FIG. 1. In that case, a score of 1-2 is indicative of a minimum susceptibility to an agent which target the PD-1/PD-L1 pathway, a score of 3-5 is indicative of a moderate susceptibility to an agent which targets the PD-1/PD-L1 pathway and a score in excess of 6 is indicative of susceptibility to an agent which targets the PD-1/PD-L1 pathway.

Suitably, the algorithm is integrated into the high throughput system used to derive the biomarker data, so that the results are generated. Such systems will comprise a processor and a memory storing instructions to receive the data obtained using the method of the invention, analyse and transform it to produce a ‘score’ indicative of the susceptibility of the patient to treatment using an agent which targets a component of the PD-1/PD-L1 pathway using the algorithm described above. These results may then be suitably displayed on a graphic interface. In some cases, the memory will comprise a non-transitory computer-readable medium. Such systems and mediums form a further aspect of the invention.

Genetic variants detected using the method of the invention may be linked via a suitable bioinformatics platform to a wide range of potential agents which target the PD-1/PD-L1 pathway from those in clinical trials through to FDA/EMA approved therapies.

Once identified in this way, patients whose tumours are susceptible to such inhibitors may be treated accordingly, using suitable agents. Thus in a further aspect, the invention provides a method for treating a patient suffering from proliferative disease, said method comprising carrying out a method as described above using a tumour sample from said patient, developing a customized recommendation for treatment or continued treatment of the patient, based an analysis of the biomarkers, and administering a suitable therapy or treatment to said patient. In particular, those patients who are identified as being susceptible or highly susceptible to treatment using agents which target components of the PD-1/PD-L1 pathway may be treated with such agents, while those identified as having little susceptibility using the algorithm will be treated with alternative agent types. These will be administered in line with normal clinical practice.

Thus, in a particular embodiment, the method of the invention further comprises generating a customised recommendation for treatment, based upon the results obtained. Integrating information derived from such mutations allows for a customised recommendation for therapy to be prepared using for example the systems an mediums described above, and in such cases, these may also be displayed on a graphical interfact.

The method of the invention addresses the problems and severe limitations of current IHC based companion diagnostics for anti-PD-1/PD-L1/PD-L2 directed immunotherapies. In particular, the method is amenable for full automation. It may be quantitative in particular when using the algorithm described above, and does not require subjective human interpretation by a pathologist. In particular embodiments, the method of the invention does not require the input of a pathologist for manual assessment of PD-L1 expression. The whole test is fully automated and therefore is not subject to inter-observer or intra-observer variability.

In a particular embodiment, the method of the invention provides a comprehensive and integrated readout of all biomarkers linked to response to anti-PD-1/PD-L1/PD-L2 immunotherapies. The algorithm, as described above, can be used to integrate all these predictive biomarkers into a Polygenic Predictive Score (PPS) (FIG. 1). As discussed above, a PPS score of 0 indicates no response to anti-PD-1/PD-L1/PD-L2 immunotherapies. A score of 1-2 is indicative of minimal response; a score of 3-5 indicates moderate response and a score of 6-8 is indicative of major response.

This quantitative nature of the assay means precise cut points can be identified that predict response to anti-PD-1/PD-L1/PD-L2 directed immunotherapies. By providing a clear quantitative result, any subjectivity and inter-observer variability associated interpretation of IHC based assays is avoided.

The method of the invention is able to assess the complete PD-1/PD-L1 signalling axis in an integrated approach which cannot be achieved using a single IHC biomarker. Using the method of the invention, activation/increased expression of many elements in the PD-1/PD-L1 signalling axis including PD-1, PD-L1, PD-L2, NFATC1, PIK3CA, PIK3CD, PRDM1, PTEN, PTPN11, MTOR, HIF1A, IFN-gamma and FOXO1 may be assessed.

Critically, the method of the invention may include assessment of oncogenic mutations that are linked to response to anti-PD-1/PD-L1/PD-L2 directed immunotherapies. A broad range of oncogenes are assessed for gene aberration, mutations, fusions and amplification (Tables 3 and FIG. 1 A) with linkage to anti-PD-1/PD-L1 directed immunotherapies. Many of these genes are components of growth signalling networks. Oncogenic activation of these growth signalling networks leads to induction of the PD-1 ligands PD-L1 and PD-L2.

Thus the method of the invention may provide a fully automated test that has been designed to analyse all components involved in the PD-1/PD-L1 immune regulatory anti-cancer response including tumour cells, T immune cells and antigen presenting cells (APSs). This provides a quantitative integrated picture of all components involved in the PD-1/PD-L1/PD-L2 immune regulatory cancer response in terms of all cell types (tumour cell and immune cells) and at all levels of the PD-1/PD-L1 signalling axis.

In addition to a comprehensive analysis of the PD-1/PD-L1/PD-L2 signalling axis, the method of the invention has been designed to detect all oncogenic mutations that impact on response to anti-PD-1/PD-L1 directed immunotherapies as described above. Integrating information derived from the PD-1/PD-L1/PD-L2 signalling axis with oncogenic predictors provides the most powerful predictor of response to anti-PD-1/PD-L1/PD-L2 directed immunotherapies. For example detection of (i) mutated and (ii) amplified PD-L1 is also linked to (iii) increased expression of PD-L1. This provides three independent but linked predictors of response to anti-PD-1/PD-L1/PD-L2 directed immunotherapies. By integrating information from the PD-1/PD-L1/PD-L2 signalling axis with oncogenic activation of growth signalling networks, the algorithm described herein is able to precisely identify those patients most likely to respond to anti-PD-1/PD-L1/PD-L2 directed immunotherapies (Tables 3A-D and FIG. 1).

The method of the present invention can use high throughput analytical platforms to match patient's tumours to specific targeted therapies, from FDA/EMA approved, ESMO/NCCN guideline references and in all phases of clinical trials worldwide.

The applicants have utilised the Thermo Fisher Ion Torrent platform to develop the assay of the invention. The aim was, for the reasons explained above, to provide a comprehensive picture of the PD-1/PD-L1 signalling axis by analysing many components of this pathway as outlined above. Use of a single integrated test as described herein, and using sets of primers spanning the exon/intron boundaries of both immune regulatory genes at multiple loci across the both genes and has been designed in such a way that they are able to amplify the degraded RNA material extracted from routine formalin fixed wax embedded clinical tumour material/biopsy/resection specimens. The accurate and precise measurement of these multiple components of the PD-1/PD-L1/PD-L2 signalling axis allows for the provision of a quantitative integrated profile of this immune regulatory pathway. The output from this assay platform can then be used to provide precise cut offs for these immune regulatory biomarkers which are predictive of therapeutic response to anti-PD-1/PD-L1/PD-L2 directed therapies. Amplicons have also been designed to overlap for sequence coverage redundancy to optimise amplification of fragmented DNA templates obtained from routine diagnostic PWET samples. The DNA analysis is designed to detect oncogenic mutations and gene copy aberrations which have been identified as predictors of response to anti-PD-1/PD-L1/PD-L2 directed immunotherapies. In addition to RNA expression analysis of PD-1/PD-L1/PD-L2 signalling molecules RNA expression analysis if performed to detect oncogenic fusion transcripts identified as predictors of response to anti-PD-1/PD-L1/PD-L2 directed immunotherapies.

Kits suitable for carrying out the method of the invention are novel and form a further aspect of the invention. These may comprise combinations of amplification primers required to detect 3 or more of the biomarkers listed in Table 3.

Furthermore, apparatus arranged to carry out the method described above are also novel and form a further aspect of the invention. In particular, apparatus will comprise means for carrying out DNA and/or RNA analyses as described above, linked to a computer programmed to implement the algorithm as described above. A computer or a machine-readable cassette programmed in this way forms yet a further aspect of the invention, as do systems and non-transitory computer-readable mediums which allow for the method described herein to be carried.

### DETAILED DESCRIPTION OF THE INVENTION

The invention will now be particularly described by way of example with reference to the accompanying diagrammatic drawings in which:

FIG. 1 is a schematic diagram illustrating a method embodying the invention including an algorithm used to interpret the results and provide an indicate of patient susceptibility.

### EXAMPLE 1

### Materials and Methods

### Overview of Primer Design:

Primers for detecting each of the biomarkers listed in Tables 3A-3D were designed in accordance with conventional practice. In general, primer of 18-30 nucleotides in length are optimal with a melting temperature (Tₘ) between 65° C. 75° C. The GC content of the primers should be between 40-60%, with the 3′ of the primer ending in a C or G to promote binding. The formation of secondary structures within the primer itself is minimised by ensuring a balanced distribution of GC-rich and AT-rich domains. Intra/inter primer homology should be avoided for optimal primer performance.

### Primers for Copy Number Detection:

Primers were designed to span precise regions in the genes listed in table 3D with several amplicons per gene. The depth of coverage is measured for each of these amplicons. The copy number amplification and deletion algorithm is based on a hidden Markov model (HMM). Prior to copy number determination, read coverage is corrected for GC bias and compared to a preconfigured baseline.

### Primer for Hotspot Detection:

Primers were designed to target specific regions prone to oncogenic somatic mutations as listed in Table 3B and in consideration with the general points discussed above.

### Primers for RNA Expression Analysis:

Extracted RNA is processed via RT-PCR to create complementary DNA (Cdna) which is then amplified using specific primers. Multiple primer sets were designed to span the exon/intron boundaries across all genes subject to expression analysis as listed Table 3A.

### Primers for RNA Fusion Detection:

A pair of targeted exon-exon breakpoint assay primers are designed for each fusion listed in Table 3C. Primers flanking the fusion breakpoint generate specific fusion amplicons which are aligned to the reference sequence allowing for identification of fusion genes. Expression imbalance assays enable the equivalent expression levels to be monitored in normal samples, with an imbalance between the 5′ and 3′ assays indicating samples have a fusion breakpoint.

An algorithm has been developed to integrate information derived from this targeted genomic analysis of routine paraffin wax embedded tissue biopsy specimens to predict response to anti-PD-1/PD-L1/PD-L2 directed immunotherapies as shown in FIG. 1.

### DNA and RNA Extraction

DNA and RNA was extracted from FFPE curls cut at 10 μm or from sections on unstained slides at 5 μm using the RecoverAll™ Ambion extraction kit (Cat no.A26069). Two Xylene washes were performed by mixing 1 ml of xylene with the sample. The samples were centrifuged and xylene removed. This was followed by 2 washes with 1 ml of pure ethyl alcohol. After the samples 1.5 were air-dried, 25 μl of digestion buffer, 75 μl of nuclease free water and 41 μl of protease were added to each sample. Samples were then digested at 55° C. for 3 hours followed by 1 hour digestion at 90° C. 120 μl of Isolation additive was mixed with each sample and the samples added to filter cartridges in collection tubes and centrifuged. The filters were moved to new collection tubes and kept in the fridge for DNA extraction at a later stage. The flow-through was kept for RNA extraction and 275 μl of pure ethyl alcohol was added and the sample moved to a new filter in a collection tube and centrifuged. After a wash of 700 μl of Wash 1 buffer the RNA was treated with DNase as follows; a DNase mastermix was prepared using 6 μl of 10× DNase buffer, 50 μl of nuclease free water and 4 μl of DNase per sample. This was added to the centre of each filter and incubated at room temperature for 30 minutes.

After the incubation 3 washes were performed using Wash 1, then Wash 2/3 removing the wash buffer from the collection tubes after each centrifugation. The filters were moved to a new collection tube and the elution solution (heated to 95° C.) was added to each filter and incubated for 1 minute. After centrifuging the sample, the filter was discarded and the RNA collected in the flow through moved to a new low bind tube.

The DNA in the filters were washed with Wash 1 buffer, centrifuged and flow through discarded. The DNA was treated with RNase (50 μl nuclease water and 10 μl RNase) and incubated at room temperature for 30 minutes. As above with the RNA, three washes were completed and the samples eluted in elution solution heated at 95° C.

### DNA and RNA Measurement

The quantity of DNA and RNA from the extracted samples were measured using the Qubit® 3.0 fluorometer and the Qubit® RNA High Sensitivity Assay kit (CAT: Q32855) and Qubit® dsDNA High Sensitivity Assay kit (Cat:Q32854). 1 μl of RNA/DNA combined with 199 μl of combined HS buffer and reagent were used in qubit assay tubes for measurement. 10 μl of standard 1 or 2 were combined with 190 μl of the buffer and reagent solution for the controls.

### Library Preparation

RNA samples were diluted to 5 ng/μl if necessary and reverse transcribed to cDNA in a 96 well plate using the Superscript Vilo cDNA synthesis kit (CAT 11754250). A mastermix of 2 μl of vilo, 1 μl of 10× SuperScript III Enzyme mix and 5 μl of nuclease free water was made for all of the samples. 8 μl of the mastermix was used along with 2 μl of the RNA in each well of a 96 well plate. The following program was run:

| Temperature | Time | |
|---|---|---|
| 42° C. | 30 | min |
| 85° C. | 5 | min |

| | |
|---|---|
| 10° C. | Hold |

Amplification of the cdna was then performed using 4 μl of 6 RNA primers covering multiple exon-intron loci across the gene, 4 μl of Ampliseq Hi-Fi*¹ and 2 μl of nuclease free water into each sample well. The plate was run on the thermal cycler for 30 cycles using the following program: *1 From the Ion Ampliseg™ library 2.0 (Cat:4480441)*2 From the Ion 540™ OT2 kit (Cat: A27753)

| Stage | Step | Temperature | Time | |
|---|---|---|---|---|
| Hold | Activate the enzyme | 99° C. | 2 | min |
| Cycle (30 | Denature | 99° C. | 15 | sec |
| cycles) | Anneal and extend | 60° C. | 4 | min |

| | | | |
|---|---|---|---|
| Hold | — | 10° C. | Hold |

DNA samples were diluted to 5 ng/μl and added to Ampliseq Hi-fi*¹, nuclease free water and set up using two DNA primer pools (5 μl of pool 1 and 5 μl of pool 2) in a 96 well plate. The following program was run on the thermal cycler:

| Stage | Step | Temperature | Time | |
|---|---|---|---|---|
| Hold | Activate the enzyme | 99° C. | 2 | min |
| Cycle (18 | Denature | 99° C. | 15 | sec |
| cycles) | Anneal and extend | 60° C. | 4 | min |

| | | | |
|---|---|---|---|
| Hold | — | 10° C. | Hold (up to 16 hours) |

Following amplification, the amplicons were partially digested using 2 μl of LIB Fupa*¹, mixed well and placed on the thermal cycler on the following program:

| Temperature | Time |
|---|---|
| 50° C. | 10 min |
| 55° C. | 10 min |
| 60° C. | 20 min |
| 10° C. | Hold (for up to 1 hour) |

4 μl of switch solution*¹, 2 μl of diluted Ion XPRESS Barcodes 1-16 (CAT: 4471250) and 2 μl of LIB DNA ligase*¹ were added to each sample, mixing thoroughly in between addition of each component. The following program was run on the thermal cycler:

| Temperature | Time |
|---|---|
| 22° C. | 30 min |
| 72° C. | 10 min |
| 10° C. | Hold (for up to 1 hour) |

The libraries were then purified using 30 μl of Agencourt AMPure XP (Biomeck Coulter cat: A63881) and incubated for 5 minutes. Using a plate magnet, 2 washes using 70% ethanol were performed. The samples were then eluted in 50 μl TE.

qPCR

The quantity of library was measured using the Ion Library Taqman quantitation kit (cat: 4468802). Four 10-fold serial dilutions of the E. coli DH10B Ion control library were used (6.8 pmol, 0.68 pmol, 0.068 pmol and 0.0068 pmol) to create the standard curve. Each sample was diluted 1/2000, and each sample, standard and negative control were tested in duplicate. 10 μl of the 2× Taqman mastermix and 1 μl of the 20× Taqman assay were combined in a well of a 96 well fast thermal cycling plate for each sample. 9 μl of the 1/2000 diluted sample, standard or nuclease free water (negative control) were added to the plate and the qPCR was run on the ABI StepOnePlUS™ machine (Cat: 4376600) using the following program:

| Stage | Temperature | Time | |
|---|---|---|---|
| Hold (UDG incubation) | 50° C. | 2 | min |
| Hold (polymerase activation) | 95° C. | 20 | sec |
| Cycle (40 cycles) | 95° C. | 1 | sec |
| | 60° C. | 20 | sec |

Samples were diluted to 100 pmol using TE and 10 μl of each sample pooled to either a DNA tube or RNA tube. To combine the DNA and RNA samples, a ratio of 80:20 DNA:RNA was used.

### Template Preparation

The Ion One Touch™ 2 was initialized using the Ion S5 OT2 solutions and supplies*² and 150 μl of breaking solution*² was added to each recovery tube. The pooled RNA samples were diluted further in nuclease free water (8 μl of pooled sample with 92 μl of water) and an amplification mastermix was made using the Ion S5 reagent mix*² along with nuclease free water, ION S5 enzyme mix*², Ion sphere particles (ISPs)^{*2} and the diluted library. The mastermix was loaded into the adapter along with the reaction oil*². The instrument was loaded with the amplification plate, recovery tubes, router and amplification adapter loaded with sample and amplification mastermix.

### Enrichment

For the enrichment process, melt off was made using 280 μl of Tween*² and 40 μl of 1M Sodium Hydroxide. Dynabeads® MyOne™ Streptavidin C1 (CAT:65001) were washed with the OneTouch wash solution*² using a magnet. The beads were suspended in 130 μl of MyOne bead capture solution*². The ISPs were recovered by removing the supernatant, transferring to a new low bind tube and subsequently washed in 800 μl of nuclease free water. After centrifuging the sample and removing the supernatant of water, 20 μl of template positive ISPs remained. 80 μl of ISP resuspension solution*² was added for a final volume of 100 μl.

A new tip, 0.2 ml tube and an 8 well strip was loaded on the OneTouch™ ES machine with the following:
Well 1: 100 μl of template positive ISPs
Well 2: 130 μl of washed Dynabeads® MyOne™ streptavidin C1 beads, resuspended in MyOne bead capture
Well 3: 300 μl of Ion OneTouch ES Wash solution*²
Well 4: 300 μl of Ion OneTouch ES Wash solution
Well 5: 300 μl of Ion OneTouch ES Wash solution

### Well 6: Empty

Well 7: 300 μl of melt off

### Well 8: Empty

Following the run which takes approximately 35 minutes, the enriched ISPs were centrifuged, the supernatant removed and washed with 200 μl of nuclease free water. Following a further centrifuge step and supernatant removal, 10 μl of ISPs remained. 90 μl of nuclease free water was added and the beads were resuspended.

### Sequencing

The Ion S5 System™ (Cat: A27212) was initialized using the Ion S5 reagent cartridge, Ion S5 cleaning solution and Ion S5 wash solutions*².

5 μl of Control ISPs*² were added to the enriched sample and mixed well. The tube was centrifuged and the supernatant removed to leave the sample and control ISPs. 15 μl of Ion S5 annealing buffer*² and 20 μl of sequencing primer*² were added to the sample. The sample was loaded on the thermal cycler for primer annealing at 95° C. for 2 minutes and 37° C. for 2 minutes. Following thermal cycling, 10 μl of Ion S5 loading buffer*² was added and the sample mixed.

50% annealing buffer was made using 500 μl of Ion S5 annealing buffer*² and 500 μl of nuclease free water*².

The entire sample was then loaded into the loading port of an Ion 540™ chip (Cat: A27766) and centrifuged in a chip centrifuge for 10 minutes.

Following this, 100 μl of foam (made using 49 μl of 50% annealing buffer and 1 μl of foaming solution*²) was injected into the port followed by 55 μl of 50% annealing buffer into the chip well, removing the excess liquid from the exit well. The chip was centrifuged for 30 seconds with the chip notch facing out. This foaming step was repeated.

The chip was flushed twice using 100 μl of flushing solution (made using 250 μl of isopropanol and 250 μl of Ion S5 annealing buffer) into the loading port, and excess liquid removed from the exit well. 3 flushes with 50% annealing buffer into the loading port were then performed. 60 μl of 50% annealing buffer was combined with 6 μl of Ion S5 sequencing polymerase*². 65 μl of the polymerase mix was then loaded into the port, incubated for 5 minutes and loaded on the S5 instrument for sequencing which takes approximately 3 hours and 16 hours for data transfer.

### Data Analysis

### DNA CNV Analysis:

Copy number variations (CNVs) represent a class of variation in which segments of the genome have been duplicated (gains) or deleted (losses). Large, genomic copy number imbalances can range from sub-chromosomal regions to entire chromosomes.

Raw data were processed on the Ion S5 System and transferred to the Torrent Server for primary data analysis performed using the Oncomine Comprehensive Assay Baseline v2.0. This plug-in is included in Torrent Suite Software, which comes with each Ion Torrent™ sequencer. Copy number amplification and deletion detection was performed using an algorithm based on a hidden Markov model (HMM). The algorithm uses read coverage across the genome to predict the copy-number. Prior to copy number determination, read coverage is corrected for GC bias and compared to a preconfigured baseline.

The median of the absolute values of all pairwise differences (MAPD) score is reported per sample and is used to assess sample variability and define whether the data are useful for copy number analysis. MAPD is a per-sequencing run estimate of copy number variability, like standard deviation (SD). If one assumes the log 2 ratios are distributed normally with mean 0 against a reference a constant SD, then MAPD/0.67 is equal to SD. However, unlike SD, using MAPD is robust against high biological variability in log 2 ratios induced by known conditions such as cancer. Samples with an MAPD score above 0.5 should be carefully reviewed before validating CNV call.

The results from copy number analysis after normalisation can be visualised from the raw data.

Somatic CNV detection provides Confidence bounds for each Copy Number Segment. The Confidence is the estimated percent probability that Copy Number is less than the given Copy Number bound. A lower and upper percent and the respective Copy Number value bound are given for each CNV. Confidence intervals for each CNV are also stated, and amplifications of a copy number >6 with the 5% confidence value of ≥4 after normalization and deletions with 95% CI≤4 are classified as present.

### DNA Hotspot Analysis:

Raw data were processed on the Ion S5 System and transferred to the Torrent Server for primary data analysis performed using the custom workflow. Mapping and alignment of the raw data to a reference genome is performed and then hotspot variants are annotated in accordance with the BED file. Coverage statistics and other related QC criteria are defined in a vcf file which includes annotation using a rich set of public sources. Filtering parameters can be applied to identify those variants passing QC thresholds and these variants can be visualised on IGV. In general, the rule of classifying variants with >10% alternate allele reads, and in >10 unique reads are classified as ‘detected’.

Several in-silico tools are utilised to assess the pathogenicity of identified variants these include PhyloP, SIFT, Grantham, COSMIC and PolyPhen-2.

### RNA Expression Analysis:

Raw data were processed on the Ion S5 System and transferred to the Torrent Server for primary data analysis performed using the AmpliSeqRNA plug-in. This plug-in is included in Torrent Suite Software, which comes with each Ion Torrent™ sequencer. The AmpliSeqRNA plugin uses the Torrent Mapping Alignment Program (TMAP). TMAP is optimized for Ion Torrent™ sequencing data for aligning the raw sequencing reads against a custom reference sequence set containing all transcripts targeted by the AmpliSeq kit. The assay specific information is contained within a bespoke BED file. To maintain specificity and sensitivity, TMAP implements a two-stage mapping approach. First, four alignment algorithms, BWA-short, BWA-long, SSAHA, and Super-maximal Exact Matching we employed to identify a list of Candidate Mapping Locations (CMLs). A further aligning process is performed using the Smith Waterman algorithm to find the final best mapping. As part of the ampliSeqRNA plugin, raw read counts of the targeted genes is performed using samtools (samtools view-c-F 4-L bed_file bam_file). Ion AmpliSeq RNA normalization for a given sample is automatically calculated by the plug-in as the number of reads mapped per gene per million reads mapped or RPM. This figure is then log 2-transformed normalized reads per million, (nRPM).

The bespoke BED file is a formatted to contain the nucleotide positions of each amplicon per transcript in the mapping reference. Reads aligning to the expected amplicon locations and meeting filtering criteria such as minimum alignment length are reported as percent “valid” reads. “Targets Detected” is defined as the number of amplicons detected (≥10 read counts) as a percentage of the total number of targets.

After mapping, alignment and normalization, The AmpliSeqRNA plug-in provides data on QC metrics, visualization plots, and normalized counts per gene that corresponds to gene expression information that includes a link to a downloadable file detailing the read counts per gene in a tab-delimited text file. The number of reads aligning to a given gene target represents an expression value referred to as “counts”. This Additional plug-in analyses include output for each barcode of the number of genes (amplicons) with at least 1, 10, 100, 1,000, and 10,000 counts to enable determination of the dynamic range and sensitivity per sample.

A summary table of the above information, including mapping statistics per barcode of total mapped reads, percentage on target, and percentage of panel genes detected (“Targets Detected”) is viewable in Torrent Suite Software to quickly evaluate run and library performance.

### Defining Cut Off Points for PD-1 and PD-1 Expression:

PD-1 and PD-L1 RNA expression values were determined using a range of normal tissue samples representing each tissue organ system (e.g. brain, lung, breast, colon, ovary, prostate, bladder) and correlated with PD-1 and PD-L1 protein expression levels using the Immunofocus test which utilises an immunohistochemical assay using a rabbit monoclonal antibody (E1L3, Cell signalling). These data provided a base line PD-1 and PD-L1 RNA expression levels for normal tissues in the range 0-500 nRPM. PD-1 and PD-L1 RNA expression were then determined for a range of tumour types and correlated with PD-1 and PD-L1 Immunofocus protein expression levels. Tumours showing absence or low levels of PD-1/PD-L1 RNA expression as observed in normal tissue (i.e. 0-500 nRPM) correlated with PD-1/PD-L1 tumour proportion score as determined by Immunofocus of <1%. Tumour showing high tumour proportion score assessed by Immunofocus of >50% correlated with high RNA expression in the range 1500-2000 nRPM. For tumour proportion scores between 1% and 50% a corresponding increase in PD-1 and PD-L1 RNA expression was observed in the range 500-1500 nRPM. These data conclusively show that clinically relevant levels of PD-1 and PD-L1 RNA expression can be measured by a quantitative assay and therefore able to replace the semi-quantitative and subjective IHC based assays currently used as companion diagnostic for anti-PD-1 and anti-PD-L1 directed therapies. This provides a powerful and innovative new approach to precisely identify those patients who could benefit from immunotherapeutic agents.

TABLE 1

| | | | | IHC Diagnostic Antibody | | Positivity |
|---|---|---|---|---|---|---|
| Drug | Manufacturer | Status | Target | Clone/Staining Platform | Results | Criteria |
| Pembrolizumab | Merck | Approved | PD-1 | NSCLC, metastatic melanoma | Dako 22C3/Link 48 | ≥50% tumour |
| (Keytruda) | | | | | Autostainer | cells |
| Nivolumab (Opdivo) | BMS | Approved | PD-1 | NSCLC, metastatic melanoma, | Dako 28-8/Link 48 | ≥1% tumour |
| | | | | renal cell carcinoma | Autostainer | cells |
| Atezolizumab | Roche | Phase 3 | PD-L1 | NSCLC, metastatic melanoma, | Ventana SP142/ | Tumour cells |
| (MPDL3280A | | | | renal cell carcinoma | Benchmark | and/or tumour |
| | | | | | | infiltrating cells |
| Avelumab | Pfizer | Phase 3 | PD-L1 | Solid tumours | In development with Dako | |
| (MSB00107 18C) | | | | | | |
| Durvalumab | Astra-Zeneca | Phase 2 | PD-L1 | Solid tumours | Ventana SP263/ | ≥25% tumour |
| (MEDI4736) | | | | | Benchmark | cells |
| 4 PDR001 | Novartis | Phase 2 | PD-1 | Solid tumours | Unknown | |
| BGB-A317 | BeiGene | Phase 1 | PD-1 | Solid tumours | Unknown | |
| REGN2810 | Regeneron | Phase 1 | PD-1 | Solid tumours | Unknown | |
| SHR-1210 | Incyte/ | Phase 1 | PD-1 | Solid tumours | Unknown | |
| | Hengrui | | | | | |

IHC = immunohistochemistry;
PD-1 = programmed cell death protein;
PDL-1 = programmed death ligand 1;
NSCLC = non-small cell lung cancer

TABLE 2
VENTANA PD-L1 (SP142) Assay Stepwise Scoring Algorithm for NSCLC

| | |
|---|---|
| Step 1: Tumour Cell (TC) Staining Assessment | PD-L1 Expression |
| Presence of discernible PD-L1 membrane staining of any intensity | ≥50% TC |
| in ≥50% of tumour cells | |
| Absence of any discernible PD-L1 staining-or-Presence of discernible | Proceed to Step 2 |
| PD-L1 membrane staining of any intensity in <50% of tumour cells | |
| Step 2: Tumour Infiltrating Immune Cell (IC) Staining Assessment | PD-L1 Expression |
| Presence of discernible PD-L1 staining of any intensity in tumour- | ≥10% TC |
| infiltrating immune cells covering ≥10% of tumour area occupied by | |
| tumour cells, associated intratumoural and contiguous peritumoural | |
| stroma | |
| Absence of any discernible PD-L1 staining-or-Presence of discernible | <50% TC and |
| PD-L1 staining of any intensity in tumour-infiltrating immune cells | <10% IC |
| covering <10% of tumour area occupied by tumour cells, associated | |
| intratumoural and contiguous peritumoural stroma | |

Diagnostic status from PD-L1 IHC 22C3 pharmDX results

| PD-L1 | | PD-L1 IHC 22C3 pharmDx Result: |
|---|---|---|
| Expression | Staining Pattern | Report to treating physician |
| No | Partial or complete cell membrane | PD-L1 Negative for KEYTRUDA |
| Expression | staining (≥1+) in <1% of viable tumour | |
| | cells | |
| Low | Partial or complete cell membrane | PD-L1 Negative for KEYTRUDA |
| Expression | staining (≥1+) in ≥1-49% of viable | |
| | tumour cells | |
| High | Partial or complete cell membrane | PD-L1 Positive for KEYTRUDA |
| Expression | staining (≥1+) in ≥50% of viable | |
| | tumour cells | |

TABLE 3A
Biomarkers based upon aberrant RNA Expression

| | Levels that would | | |
|---|---|---|---|
| | be regarded as | | |
| Biomarker | aberrant | Therapy | Reference |
| CD273 (PDL2) | >500 nRPM | Pembrolizumab, atezolizumab, | NCT02644369, |
| | | avelumab, nivolumab, durvalumab, | NCT02304393, |
| | | PDR-001, BGB-8317, REGN2810, SHR- | NCT01772004, |
| | | 1210 | NCT02550249, |
| | | | NCT02484404, |
| | | | NCT02608268 |
| CD274 (PDL1) | >500 nRPM | pembrolizumab, atezolizumab, | NCT02644369, |
| | | avelumab, nivolumab, durvalumab, | NCT02304393, |
| | | PDR-001, BGB-8317, REGN2810, SHR- | NCT01772004, |
| | | 1210 | NCT02550249, |
| | | | NCT02484404, |
| | | | NCT02608268 |
| CD279 (PD1) | >500 nRPM | pembrolizumab, atezolizumab, | NCT02644369, |
| | | avelumab, nivolumab, durvalumab, | NCT02304393, |
| | | PDR-001, BGB-8317, REGN2810, SHR- | NCT01772004, |
| | | 1210 | NCT02550249, |
| | | | NCT02484404, |
| | | | NCT02608268 |
| MLH1 | <500 nRPM | https://www.fda.gov/newsevents/ne | |
| PMS2 | <500 nRPM | wsroom/pressannouncements/ucm56 | |
| MSH6 | <500 nRPM | 0167.htm | |
| MSH2 | >500 nRPM | | |
| NFATC1 | >500 nRPM | pembrolizumabatezolizumab, | |
| PIK3CA | >500 nRPM | avelumab, nivolumab, durvalumab, | |
| PIK3CD | >500 nRPM | PDR-001, BGB-8317, REGN2810, SHR- | |
| PRDM1 | >500 nRPM | 1210 | |
| PTEN | >500 nRPM | | |
| PTPN11 | >500 nRPM | | |
| MTOR | >500 nRPM | | |
| HIF1A | >500 nRPM | | |
| FOXO1 | >500 nRPM | | |

TABLE 3B
DNA mutations

| Biomarker | Therapy | Reference |
|---|---|---|
| ALK mutation | PBF-509, PBF-509 + PDR-001 | NCT02403193 |
| BRAF exon 15 | atezolizumab in combination | NCT02291289 |
| mutation | aldesleukin in combination | NCT02354690 |
| | ipilimumab + nivolumab | NCT02339571 |
| | pembrolizumab in combination | MK8353-010 |
| BRAF V600D | Pembrolizumab | NCCN Guidelines ® - NCCN-Melanoma |
| mutation | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| | atezolizumab in combination | NCT02291289 |
| | atezolizumab in combination | NCT02291289 |
| | ipilimumab + nivolumab | NCCN Guidelines ® - NCCN-Melanoma |
| | | [Version 1.2017]. |
| | nivolumab | NCCN Guidelines ® - NCCN-Melanoma |
| | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| | PDR-001 | NCT02404441 |
| BRAF V600E | atezolizumab in combination | NCT02291289 |
| mutation | pembrolizumab | NCCN Guidelines ® - NCCN-Melanoma |
| | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| | nivolumab | NCCN Guidelines ® - NCCN-Melanoma |
| | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| | ipilimumab + nivolumab | NCCN Guidelines ® - NCCN-Melanoma |
| | | [Version 1.2017]. |
| | nivolumab | NCCN Guidelines ® - NCCN-Melanoma |
| | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| | PDR-001 | NCT02404441 |
| BRAF V600K | atezolizumab in combination | NCT02291289 |
| mutation | pembrolizumab | NCCN Guidelines ® - NCCN-Melanoma |
| | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| | nivolumab | NCCN Guidelines ® - NCCN-Melanoma |
| | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| | ipilimumab + nivolumab | NCCN Guidelines ® - NCCN-Melanoma |
| | | [Version 1.2017]. |
| | nivolumab | NCCN Guidelines ® - NCCN-Melanoma |
| | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| | PDR-001 | NCT02404441 |
| BRAF V600R | atezolizumab in combination | NCT02291289 |
| mutation | ipilimumab + nivolumab | NCCN Guidelines ® - NCCN-Melanoma |
| | | [Version 1.2017]. |
| | nivolumab | NCCN Guidelines ® - NCCN-Melanoma |
| | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| | PDR-001 | NCT02404441 |
| | pembrolizumab | NCCN Guidelines ® - NCCN-Melanoma |
| | | [Version 1.2017]. ESMO Clinical |
| | | Practice Guidelines - ESMO-Cutaneous |
| | | Melanoma [Ann Oncol (2015) 26 |
| | | (suppl 5): v126-v132.] |
| CD274 positive | pembrolizumab | NCT02555657 |
| | pembrolizumab in combination | NCT02494583 |
| | durvalumab | NCT02273375 |
| EGFR activating | durvalumab | NCT02273375 |
| mutation | PBF-509, PBF-509 + PDR-001 | NCT02403193 |
| EGFR exon 18 | durvalumab | NCT02273375 |
| activating mutation | PBF-509, PBF-509 + PDR-001 | NCT02403193 |
| EGFR exon 19 | durvalumab | NCT02273375 |
| activating mutation | PBF-509, PBF-509 + PDR-001 | NCT02403193 |
| EGFR exon 19 | pembrolizumab in combination | NCT02364609 |
| deletion | atezolizumab + CDX-1401 | NCT02495636 |
| | atezolizumab + erlotinib | NCT02013219 |
| | durvalumab | NCT02273375 |
| | PBF-509, PBF-509 + PDR-001 | NCT02403193 |
| EGFR exon 19 | atezolizumab + CDX-1401 | NCT02495636 |
| insertion | atezolizumab + erlotinib | NCT02013219 |
| | durvalumab | NCT02273375 |
| | PBF-509, PBF-509 + PDR-001 | NCT02403193 |
| EGFR exon 19 | atezolizumab + CDX-1401 | NCT02495636 |
| sensitizing mutation | atezolizumab + erlotinib | NCT02013219 |
| | durvalumab | NCT02273375 |
| | PBF-509, PBF-509 + PDR-001 | NCT02403193 |
| EGFR exon 20 | durvalumab | NCT02273375 |
| activating mutation | PBF-509, PBF-509 + PDR-001 | NCT02403193 |
| EGFR exon 20 | durvalumab | NCT02273375 |
| insertion | PBF-509, PBF-509 + PDR-001 | NCT02403193 |
| EGFR exon 20 | durvalumab | NCT02273375 |
| mutation | PBF-509, PBF-509 + PDR-001 | NCT02403193 |
| EGFR exon 21 | durvalumab | NCT02273375 |
| activating mutation | PBF-509, PBF-509 + PDR-001 | NCT02403193 |
| EGFR exon 21 | atezolizumab + CDX-1401 | NCT02495636 |
| sensitizing mutation | atezolizumab + erlotinib | NCT02013219 |
| | durvalumab | NCT02273375 |
| | PBF-509, PBF-509 + PDR-001 | NCT02403193 |
| EGFR G719 mutation | afatinib + pembrolizumab | NCT02364609 |
| | atezolizumab + CDX-1401 | NCT02495636 |
| | atezolizumab + erlotinib | NCT02013219 |
| | durvalumab | NCT02273375 |
| | PBF-509, PBF-509 + PDR-001 | NCT02403193 |
| EGFR L858R mutation | afatinib + pembrolizumab | NCT02364609 |
| | atezolizumab + CDX-1401 | NCT02495536 |
| | atezolizumab + erlotinib | NCT02013219 |
| | durvalumab | NCT02273375 |
| | PBF-509, PBF-509 + PDR-001 | NCT02403193 |
| EGFR L861 mutation | durvalumab | NCT02273375 |
| | PBF-509, PBF-509 + PDR-001 | NCT02403193 |
| EGFR L861Q mutation | afatinib + pembrolizumab | NCT02364609 |
| | atezolizumab + CDX-1401 | NCT02495636 |
| | atezolizumab + erlotinib | NCT02013219 |
| | durvalumab | NCT02273375 |
| | PBF-509, PBF-509 + PDR-001 | NCT02403193 |
| EGFR resistance | durvalumab | NCT02273375 |
| mutation | PBF-509, PBF-509 + PDR-001 | NCT02403193 |
| EGFR S768 mutation | atezolizumab + CDX-1401 | NCT02495536 |
| | atezolizumab + erlotinib | NCT02013219 |
| | durvalumab | NCT02273375 |
| | PBF-509, PBF-509 + PDR-001 | NCT02403193 |
| EGFR T790M | durvalumab | NCT02273375 |
| mutation | EGF-81S + nivolumab | NCT02323126 |
| | PBF-509, PBF-509 + PDR-001 | NCT02403193 |
| ERBB2 positive | atezolizumab in combination | NCT02605915 |
| FGFR aberration | durvalumab + ibrutinib | NCT02403271 |
| | durvalumab + trastuzumab | NCT02649686 |
| | pembrolizumab in combination | NCT02393248 |
| | nivolumab + sunitinib malate | NCT02400385 |
| | nivolumab + sunitinib malate | NCT02400385 |
| | pembrolizumab in combination | MK8353-010 |
| MET activating | capmatinib + nivolumab | NCT02323126 |
| mutation | | |
| KIT activating | nivolumab + sunitinib malate | NCT02400385 |
| mutation | | |
| KIT resistance | nivolumab + sunitinib malate | NCT02400385 |
| mutation | | |
| KRAS activating | Pembrolizumab in combination | |
| mutation | | |
| NRAS activating | pembrolizumab in combination | MK8353-010 |
| mutation | | |
| MET activating | Capmatinib + nivolumab | |
| mutation | | |
| MLH1 | Pembrolizumab, nivolumab, | FDA Approval 2017 |
| | atezolizumab, durvalumab | |
| MSH2 | Pembrolizumab, nivolumab, | FDA Approval 2017 |
| | atezolizumab, durvalumab | |
| MSH6 | Pembrolizumab, nivolumab, | FDA Approval 2017 |
| | atezolizumab, durvalumab | |
| PMS2 | Pembrolizumab, nivolumab, | FDA Approval 2017 |
| | atezolizumab, durvalumab | |
| POLE | Pembrolizumab, nivolumab, | |
| | atezolizumab, durvalumab | |

TABLE 3C
Gene Fusions

| Biomarker | Therapy | Reference |
|---|---|---|
| ALK fusion | alectinib + atezolizumab | NCT02013219 |
| | atezolizumab + CDX-1401 | NCT02495636 |
| | avelumab + lorlatinib | NCT02584634 |
| | ceritinib + nivolumab | NCT02393625 |
| | crizotinib + pembrolizumab | NCT02511184 |
| | nivolumab + plinabulin | NCT02812667 |
| | PBF-509, PBF-509 + PDR-001 | NCT02403193 |
| FGFR1 fusion | pembrolizumab in combination | NCT02393248 |
| FGFR2 fusion | pembrolizumab in combination | NCT02393248 |
| FGFR3 fusion | pembrolizumab in combination | NCT02393248 |
| MET fusion | capmatinib + nivolumab | NCT02323126 |

TABLE 3D
DNA copy number variants

| | Normal | | |
|---|---|---|---|
| Biomarker | copy no | Therapy | Reference |
| ERBB2 | <6 | durvalumab + ibrutinib | NCT02403271 |
| amplification | | atezolizumab in combination | NCT02605915 |
| | | durvalumab + trastuzumab | NCT02649686 |
| | | pembrolizumab + trastuzumab | NCT02129556 |
| FGFR1 | <6 | pembrolizumab in combination | NCT02393248 |
| amplification | | | |
| FGFR2 | <6 | pembrolizumab in combination | NCT02393248 |
| amplification | | | |
| FGFR3 | <6 | pembrolizumab in combination | NCT02393248 |
| amplification | | | |
| FGFR4 | <6 | pembrolizumab in combination | NCT02393248 |
| amplification | | | |
| MET | <6 | capmatinib + nivolumab | NCT02323126 |
| amplification | | | |
| CD273 (PDL2) | <6 | pembrolizumab, atezolizumab, | |
| | | avelumab, nivolumab, durvalumab, PDR-001, | |
| | | BGB-8317, REGN2810, SHR-1210 | |
| CD274 (PDL1) | <6 | pembrolizumab, atezolizumab, | |
| | | avelumab, nivolumab, durvalumab, PDR-001, | |
| | | BGB-8317, REGN2810, SHR-1210 | |

1. A method for determining the susceptibility of a patient suffering from proliferative disease to treatment using an agent which targets a component of the PD1/PD-L1 pathway, said method comprising:
a) measuring both the PD-L1 and PD-L2 copy mutation variant,
b) determining in a tumour sample from said patient at least three biomarkers, wherein at least one of the biomarker detected is directly associated with PD-1/PD-L1 pathway, at least one of the biomarkers is not directly associated with the PD1/PD-L1 pathway but is listed in Tables 3A-3D, and at least one biomarker is the tumour mutational burden, and
c) relating the overexpression of mRNA and the presence of more than one of said biomarkers as an indicator of susceptibility to said agent.

2. A method according to claim 1 wherein at least 10 of the biomarkers in Tables 3A-3D are determined.

3. A method according to claim 2 wherein all of the biomarkers in Tables 3A-3D are determined.

4. (canceled)

5. A method according to claim 1 wherein the proliferative disease is cancer.

6. (canceled)

7. A method according to claim 1 wherein the biomarker is identified using DNA or RNA analysis or a combination thereof.

8. (canceled)

9. A method according to claim 1 wherein targeted semiconductor sequencing is applied to cover the entire coding regions of the said biomarkers.

10. A method according to claim 1 wherein levels of RNA of at least one biomarker is measured, wherein a change in the expression level as compared to wild type is indicative of the PD-1/PD-L1 pathway.

11. A method according to claim 10 wherein the said biomarker is selected from the group consisting of CD273 (PD-L2), CD274 (PD-L1) and CD279 (PD-1).

12. A method according to claim 11 wherein analysis of gene expression at multiple exon-intron loci across PD-L1 and PD-1 mRNAs is carried out, and this is related to normalised gene expression so as to quantitate measurements of PD-1 and PD-L1 RNA expression levels.

13. A method of according to claim 10 wherein elevated levels of RNA of NFATC1 (Nuclear Factor Of Activated T-Cells 1), PIK3CA (Phosphatidylinositol-4,5-Bisphosphate 3-Kinase Catalytic Subunit Alpha) PIK3CD (Phosphatidylinositol-4,5-Bisphosphate 3-Kinase Catalytic Subunit Delta), PRDM1 (PR domain zinc finger protein 1), PTEN, (Phosphatase and tensin homolog), PTPN11 (Tyrosine-protein phosphatase non-receptor type 11), MTOR (mechanistic target of rapamycin), HIF1a (Hypoxia-inducible factor 1-alpha) and FOXO1m (forkhead box class 01 mutant) are determined.

14. A method any according to claim 10 wherein a loss of gene expression of the mismatch repair genes MLH1, PMS2, MSH6 and MLH2 is determined.

15. A method according to claim 1 wherein at least one biomarker comprises detection of a mutation in a gene, wherein said mutation impacts on expression or function of the gene or gene product.

16. A method according to claim 15 wherein the a mutation is present in a gene selected from the group consisting of ALK (anaplastic lymphoma kinase gene), BRAF (B-Raf gene), CD274 (PD-L1 gene), EGFR (epidermal growth factor receptor gene), ERBB2 (Receptor tyrosine-protein kinase erbB-2 or human epidermal growth factor receptor 2 gene), FGFR (fibroblast growth factor receptor gene), KIT (KIT proto-oncogene receptor tyrosine kinase gene), KRAS (K-ras gtpase gene), MET (MET proto-oncogene, receptor tyrosine kinase) or NRAS (N-ras protooncogene, GTPase gene).

17. A method according to claim 1 wherein a biomarker resulting from gene rearrangement leading to aberrant gene fusions is detected.

18. A method according to claim 17 wherein the gene fusion is a ALK fusion, an FGFR fusion of a MET gene fusion.

19. A method according to claim 1 wherein a biomarker detected comprises the presence of copy number variant of a gene.

20. A method according to claim 19 wherein the gene is selected from the group consisting of ERBB2, FGFR, FGFR1, FCFR2, FGFR3, FGFR4, CD273 (PD-L2 gene) or CD273.

21. (canceled)

22. A method according to claim 1 wherein an algorithm indicative of the level of susceptibility is applied to the results obtained.

23. A method according to claim 22 wherein a score of ‘0’ is applied to results which show no changes over wild type or normal expression profiles of the various biomarkers, whereas a score of at least 1 is applied to any mutations or variations noted.

24. A method according to claim 23, wherein
(i) a score of ‘1’ is applied in the case of the presence of an oncogenic mutation in a biomarker gene, or to the presence of 2-3 additional copies of a biomarker gene, or for the presence of an oncogenic gene fusion, or to a small (i.e. 0-500 nRPM) change in RNA expression of a biomarker gene, or for a TMB of <6 mutations/Megabase;
(ii) a score of ‘2’ is applied in the case of the presence of from 4-8 additional copies of a biomarker gene; or to intermediate (500-1500 nRPM) change in RNA expression of a biomarker gene, or for a TMB of from 6-19 mutations/Megabase;
(iii) a score of ‘3’ is applied in the case of the presence of from more than 8 additional copies of a biomarker gene; or to a high (>1500 nRPM) change in RNA expression of a biomarker gene, or for a TMB of >20 mutations/Megabase;
wherein an overall score of 1-2 is indicative of a minimum susceptibility to an agent which targets the PD-1/PD-L1 pathway, a score of 3-5 is indicative of a moderate susceptibility to such an agent and a score in excess of 6 is indicative of susceptibility to such an agent.

25.-30. (canceled)

31. A system for identifying patients suffering from proliferative disease who would respond to treatment using an agent which targets a component of the PD1/PD-L1 pathway, said system comprising:
a processor; and
a memory that stores code of an algorithm that, when executed by the processor, causes the computer system to:
receive input levels of a plurality of biomarkers selected from those listed in Tables 3A-3D identified in a sample of a tumour of a patient;
receive a further input level relating to the tumour mutational burden of nucleic acid in said sample;
analyze and transform the input levels via an algorithm to provide an output indicative of the level of susceptibility of said patient to treatment using an agent which targets a component of the PD1/PD-L1 pathway;
display the output on a graphical interface of the processor.

32. A system according to claim 31 wherein the memory further comprises code to provide a customized recommendation for the treatment of the patient, based upon the input levels.

33.-35. (canceled)

36. A method for treating a patient suffering from a proliferative disease, said method comprising carrying out a method according to claim 24 using a tumour sample from said patient, developing a customized recommendation for treatment or continued treatment, based an analysis of the biomarkers, and administering a suitable therapy or treatment to said patient.
